(19) United States
(12) Patent Application Publication
(10) Pub. No.: US 2009/0032020 A1
(43) Pub. Date: Feb. 5, 2009

(54) Gas Pressure Intensifier System for use with a Ventilator or Resuscitator

(75) Inventor: Thomas R. Kleinbeck, Raytown, MO (US)

Correspondence Address: THOMAS RAYMOND KLEINBECK, 10501 EAST 64 TERRACE, RAYTOWN, MO, 64133, US

(73) Assignee: Thomas Raymond Kleinbeck, Raytown, MO (US)

(21) Appl. No.: 11/833,130

(22) Filed: Aug. 2, 2007

(51) Int. Cl. A61M 16/00; A61N 1/00

(52) U.S. Cl. 128/204.21

(57) ABSTRACT

The present invention is directed to a system for supplying gas to a pneumatic logic controlled system such as ventilator or resuscitator. The system produces a high pressure gas stream and a low pressure gas stream from an intermediate gas pressure source. The intermediate pressure source could be any oxygen supply including a chemical oxygen generator or compressor. The high pressure gas stream has the water removed from the gas stream. The high pressure gas provides an energy source to the pneumatic logic of the ventilator or a resuscitator. The output flow of the pneumatic logic is used to control the flow of the low pressure gas by means of a proportional flow control valve. The proportional flow control valve combines the signal gas flow from the pneumatic control logic and the low pressure gas.

### CROSS-REFERENCE TO RELATED APPLICATIONS

Not Applicable

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

Various types of pneumatically controlled ventilators or resuscitators are well-know in the art. Typical designs use a high pressure gas supply with a dew point of −85 degrees Fahrenheit at operating pressures of 30 to 80 psig. In general pneumatically controlled ventilators or resuscitators are not designed to operate with a chemical oxygen generator, which may contain small amounts of water. The Society of Automotive Engineers specification AS8010 section 3.2.2 places the limit on the amount of water in a chemical oxygen generator at 20 milligrams per liter of gas at a temperature of 70 degree Fahrenheit and 760 millimeters of mercury. This corresponds to a dew point of 77 degrees Fahrenheit. The first known use of a chemical oxygen generator using sodium chlorate was a produced in Germany for aircraft during the 1940's. This unit employed a single particle filter to remove the salt particles from the oxygen gas stream resulting in a chlorine smell.

Pressure intensifiers are a common place item in many pneumatic operations. All are designed for specific applications such as; increasing the clamping force in pneumatic pressing operations, or retrieving the last usable pound of dry nitrogen from a gas bottle. These designs all vent the supply gas to atmosphere, allowing the pistons or diaphragms to return to the start of the compression stroke.

It is well known that water will condense from air when it is compressed. Common shop compressor/tank units have a water drain to circumvent the problem. When the air in a compressed air tank is expanded through an orifice the temperature will drop and additional water will condense. It is common to find a dryer on a compressed air system in an industrial environment to remove water.

No known pneumatic logic controlled ventilator or resuscitator will operate reliably with water in the supply gas. Removal of the water is critical for stable operation of any known pneumatic logic controlled equipment. In the specific case of pneumatic logic controlled ventilators or resuscitator, ice can form in the control logic of the units if the water is not removed.

Chlorate candles have been used in the past as oxygen sources for ventilator or resuscitators. Chlorate candle chemical oxygen generators operate at temperatures in excess of 212 degrees Fahrenheit. Using a desiccant such as Lithium Hydroxide, which converts to Lithium Hydroxide Monohydrate at temperatures less than 212 degrees Fahrenheit, will only delay the point water will enter the gas stream exiting the chemical generator.

Chlorate candle oxygen generator systems are typically designed for low pressure operation. When these units are employed in a high pressure system they will operate at significantly higher temperatures. Typical oxygen generators, designed for aviation use, have an exiting gas stream temperature approximately equal to room temperature. Using 78 degrees Fahrenheit, raising the pressure to 30 psig will increase the gas stream temperature to 218 degrees Fahrenheit. Further raising the pressure to 80 psig will increase the gas stream temperature to 380 degrees Fahrenheit. Choosing a stainless steel, such as Type 410, for the container of the chemical oxygen generator the yield strength is approximately is 175,000 psi. Raising operating temperature to 380 degrees Fahrenheit reduces the yield strength to 148,000 psi. This increase in temperature will require the chlorate candle container to need 15% more material for safe operation.

Chlorate oxygen generator candle are commonly manufactured as a one piece unit. If the initiation section or the main body candle does not perform adequately the entire candle is waste material.

### BRIEF SUMMARY OF THE INVENTION

It is the object of this invention to provide a method of delivering oxygen at sufficient pressure, with a sufficiently low dew point in the high pressure gas stream, to operate commercial off the shelf field transportable, emergency, or purpose designed ventilators or resuscitators, in a safe and stable manner. An example of a ventilator that can use two gas sources is the Smiths Medical, England, “Pneupac” product. This invention could be incorporated into the design of a single patient ventilator or as a stand alone unit. The invention uses a pressure intensifier to produce a high pressure gas stream and a low pressure gas stream from an intermediate pressure source. The invention may use a heat exchanger to reduce the temperature of the high pressure gas stream. The invention uses a desiccant such as lithium hydroxide, a molecular sieve, or a chemical reaction to remove water from the gas streams. This invention reduces the operating temperature and pressure of a chemical oxygen generating source used to power a pneumatic logic controlled ventilator or resuscitator. Other heat exchangers may be added to the system to reduce component operating temperatures and gas stream temperatures.

The pressure intensifier can be designed with two pistons of different diameters to gain a mechanical advantage to increase the pressure of the gas in the high pressure stream. This is the simplest implementation to design and manufacture. In this invention the gas from the intensifier that is normal vented to atmosphere is stored for later use by the ventilator or resuscitator. No known pressure intensifier system is designed to use both exiting gas streams in a process.

In order to use both streams of gas exiting the pressure intensifier a low pressure control valve is employed and operated by the pneumatic logic system. A proportional low pressure control valve controlled by a variable orifice, and powered by the pneumatic control logic was invented.

The oxygen generator designed for this system uses Hopcalite as a catalyst to react with any carbon monoxide forming carbon dioxide. The oxygen generator utilizes lithium hydroxide to react with any carbon dioxide formed. The oxygen generator contains a supper oxide to react with water and carbon dioxide.

Hopcalite is a mixture of copper and manganese oxides used as a catalyst to convert carbon monoxide into carbon dioxide when exposed to the oxygen in air. It is common to use Hopcalite in chemical oxygen generators, due to the low temperature required for the conversion reaction to take place. Hopcalite can be purchased ready to use from its manufacturer. Hopcalite granules can be used or optionally reduced to a fine powder and mixed with water. The water mixture must be drawn through a filter media and dried at 600 to 650 degree Fahrenheit.

Lithium hydroxide (LiOH) will react with carbon dioxide, from the previous reaction, through the following reaction of FIG. 14. Lithium hydroxide can be found in several off the self re-breathers and has been used in submarines since the 1930's. It can be employed either as crushed particles or applied to an appropriate filter media. In the case of oxygen stream, glass and ceramics are the most common filter medias used.

The addition of a supper oxide to the filter section of the chemical oxygen generator, such as a chlorate candle, will remove the water from the oxygen stream of the generator. The supper oxide may be manufactured into an external filter. When an oxygen stream from a chemical oxygen generator passes through the supper oxide filter bed the water will react with the supper oxide. Potassium super oxide (KO₂) will produce oxygen from water vapor (H₂O) through the following reaction of FIG. 15, which is exothermic in nature. Examples of this are the United States Navy “Oxygen Breathing Apparatus” used in onboard ship fire fighting equipment which uses moisture from exhaled breath to produce oxygen.

The chlorate candle used in the generator is assembled from components that are manufactured separately in a lot controlled manner. The two components are an initiation pellet and the main body of the candle. The outer surface along the central axis of the initiation pellet is tapered, a matching taper in manufactured into the main body of the candle. The initiation pellets are tested for performance by using a soldering iron adjusted a range of 450 to 650 degrees Fahrenheit. The main body of the chlorate candles are tested by using initiation pellets that have been qualified in the quality control process.

An auxiliary connection is provided for operation from an outside source such as high pressure bottled gas. This allows the use of the invention with an external low pressure source such as a chemical oxygen generator or compressor.

### BRIEF DESCRIPTION OF THE SEVERAL VIEWS OF THE DRAWING

FIG. 1 shows the pressure intensifier and pneumatic circuit at the end of the compression stroke. FIG. 1 is a sectional view of the intensifier. The intensifier design uses two different sizes of pistons to create a multiplier relative to the area ratio of the pistons.

The FIG. 2 shows the intensifier and circuit at the top of the compression stroke. FIG. 2 is a sectional view of the intensifier.

FIG. 3 shows the intensifier and pneumatic circuit with a heat exchanger added to the supply line added leading to the high pressure side of the intensifier. FIG. 3 is a sectional view of the intensifier. Adding a heat exchanger to the circuit reduces the operating temperatures of the components in the system. By placing the heat exchanger before the intensifier high pressure inlet check valve the operating temperature of the lower chamber of the intensifier is reduced.

FIG. 4 shows the intensifier and pneumatic circuit with a heat exchanger added to the supply line added leading to the high pressure side and low pressure side of the intensifier. FIG. 4 is a sectional view of the intensifier.

FIG. 5 shows the intensifier and pneumatic circuit with a heat exchanger added to the high pressure line exiting the intensifier. FIG. 5 is a sectional view of the intensifier. By placing the heat exchanger after the intensifier the maximum heat is transferred between the hot and cold mediums.

FIG. 6 shows the intensifier and pneumatic circuit with a heat exchanger incorporated into the design of the high pressure side of the intensifier. FIG. 6 is a sectional view of the intensifier.

FIG. 7 shows the intensifier and pneumatic circuit with the vent of the intensifier connected to a heat exchanger cooling the supply leading to the intensifier. FIG. 4 is a sectional view of the intensifier.

FIG. 8 is a diagram of the connections to the parts of the system, including intensifier, dryers, accumulators, auxiliary pressure connection and auxiliary pressure regulators.

FIG. 9 shows the intensifier constructed using a pair of diaphragms of different diameters. FIG. 9 is a sectional view of the intensifier. The advantage of the intensifier constructed with diaphragms is reduced friction since there is no moving seal in contact with the housing.

FIG. 10 shows intensifier constructed from a turbine and a centrifugal compressor. FIG. 10 is a sectional view of the intensifier. The check valves in each of the intensifier control circuit limit the flow direction in the circuit. The advantages of a turbine are a reduction in the number of moving parts and weight.

FIG. 11 is a diagram showing the high pressure gas flow from the intensifier circuit being used to power the pneumatic logic controls. The low pressure gas flow from the intensifier circuit is connected to a low pressure control valve. The low pressure control valve is proportional.

FIG. 12 is a proportional flow control valve operated by the gas flow from pneumatic control logic.

FIG. 13 is a proportional flow control valve operated by the gas flow from the pneumatic control logic with a venture tube added to the outlet of the valve.

FIG. 14 is the lithium hydroxide and carbon dioxide reaction.

FIG. 15 is the potassium supper oxide and water reaction.

FIG. 16 is an assembled chemical oxygen generator. This is a sectional view along the central axis.

FIG. 17 shows the heat shield, the chemical oxygen generator cartridge, connector fastener.

FIG. 18 shows the components of the heat shield.

FIG. 19 shows handle, fasteners, and initiation mechanism.

FIG. 20 shows the chemical oxygen can, the chemical oxygen core with insulator in place, and the exit end core locator.

FIG. 21 shows the chemical oxygen core and the insulator.

FIG. 22 shows the generator tube, initiator end core locator, and initiator end cap assembly.

FIG. 23 shows the components of the initiator end cap.

FIG. 24 shows the components of the exit end cap.

FIG. 25 shows the components of the particle filter and exit end seal.

FIG. 26 shows the components of the chemical filter of the chemical oxygen generator.

FIG. 27 shows the components of the initiator mechanism.

FIG. 28 shows the components of the chemical oxygen generator core.

FIG. 29 is a diagram of the invention showing the information of FIG. 8, FIG. 11, and the intensifier circuit; with three safety pressure relief valves added.

### DETAILED DESCRIPTION OF THE INVENTION

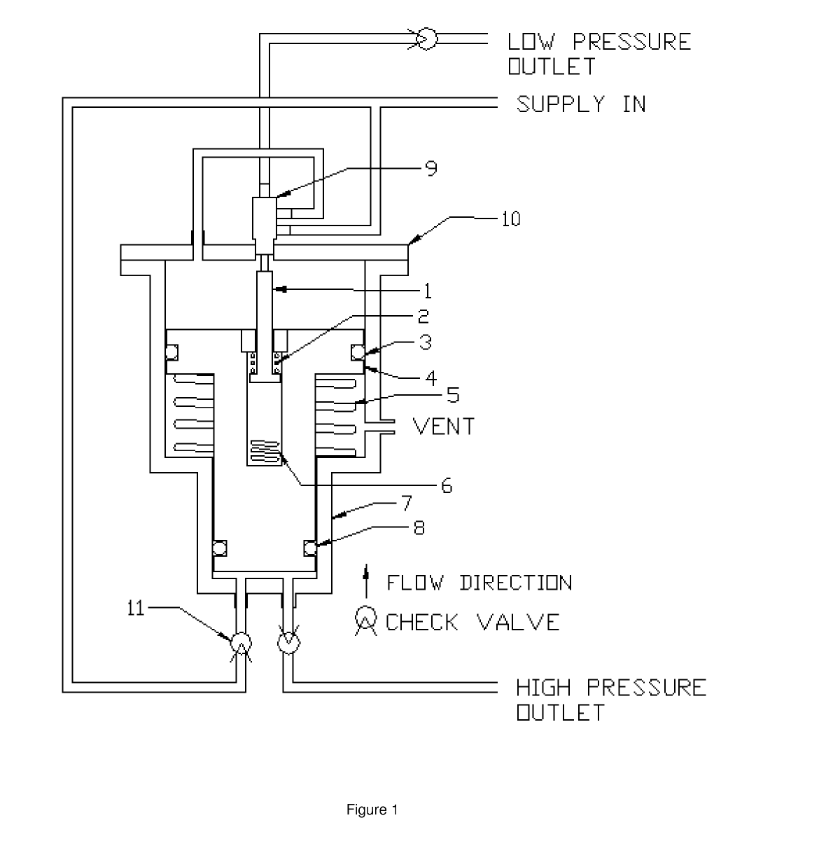

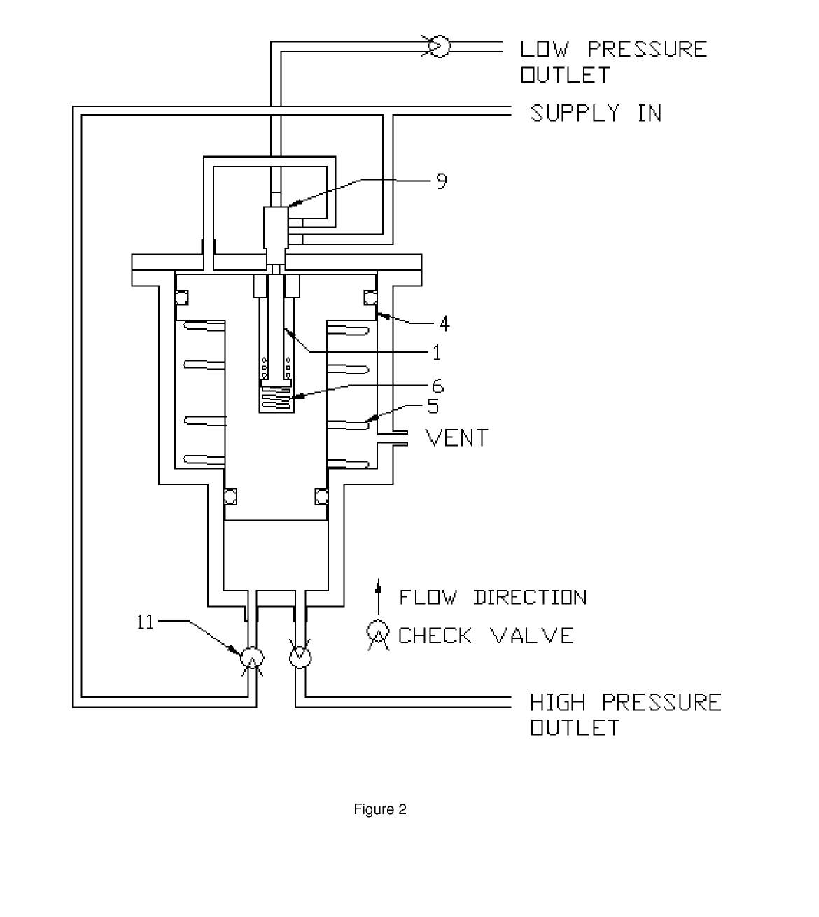

FIG. 1 shows the basic pneumatic circuit for the pressure piston type intensifier. FIG. 1 is a sectional view of the intensifier. In this particular design the intensifier uses two different piston diameters, item 3, and a cylinder, item 7, to produce a gas of higher pressure than the supply pressure. Items 3 and 8 are seals to separate gas pressures in the three chambers of the intensifier assembly. Item 1 is the flow control actuator rod; it changes the position of the 3 port valve, item 9. The control actuator rod is moved by the springs, items 2 and 6. When the piston, item 4, moves to the bottom of the cylinder the actuator rod moves pulling the 3 port valve, item 9, into a new position stopping the flow of supply gas into the large chamber and allowing the gas there to escape to the low pressure circuit. The check valves, item 11, allow the flow of gas in only one direction. Item 5 is a spring strong enough to overcome the friction of the intensifier assembly ensuring when the system has no supply gas pressure the piston will be at the top end of the intensifier assembly. Item 10 is the closure for the larger chamber of the piston type intensifier assembly.

FIG. 2 shows the piston type intensifier with the upper chamber at minimum volume. FIG. 2 is a sectional view of the intensifier. The position and orientation of the check valves allows the flow of gas to the high pressure gas when the intensifier is connected to a supply gas source. The operation of the piston will begin when the pressure at the accumulator and supply gas are equal.

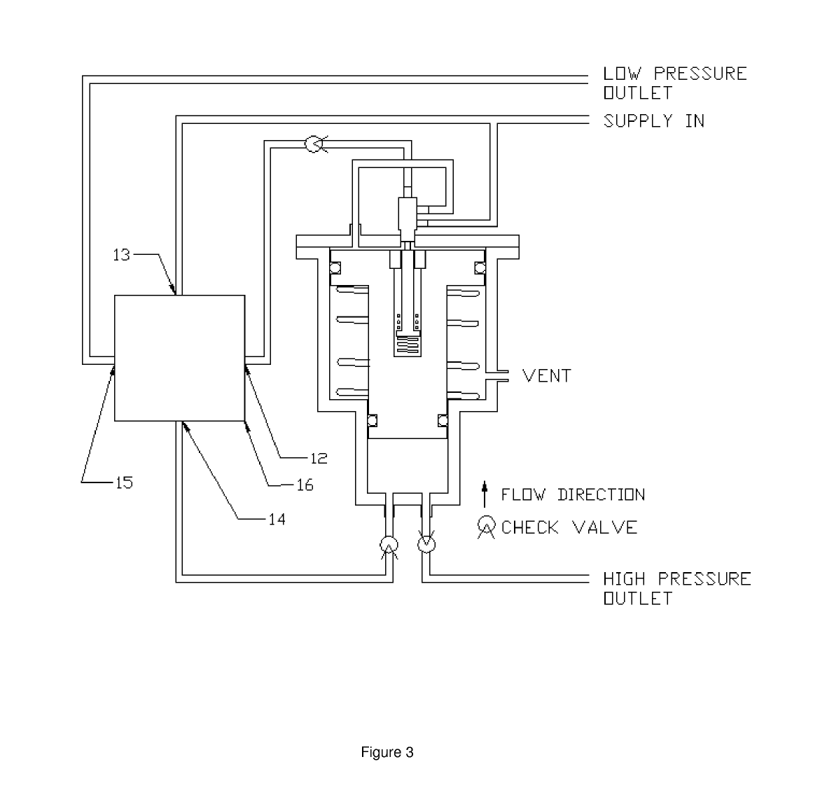

FIG. 3 show a heat exchanger, item 16, added to the circuit. FIG. 3 is a sectional view of the intensifier. The heat exchanger design is a two separate path. Item 13 is the inlet from the supply gas and item 14 is the supply gas exit. Item 12 is the gas coming from the larger chamber which is cooled by expansion, item 15 is exit of this gas from the heat exchanger.

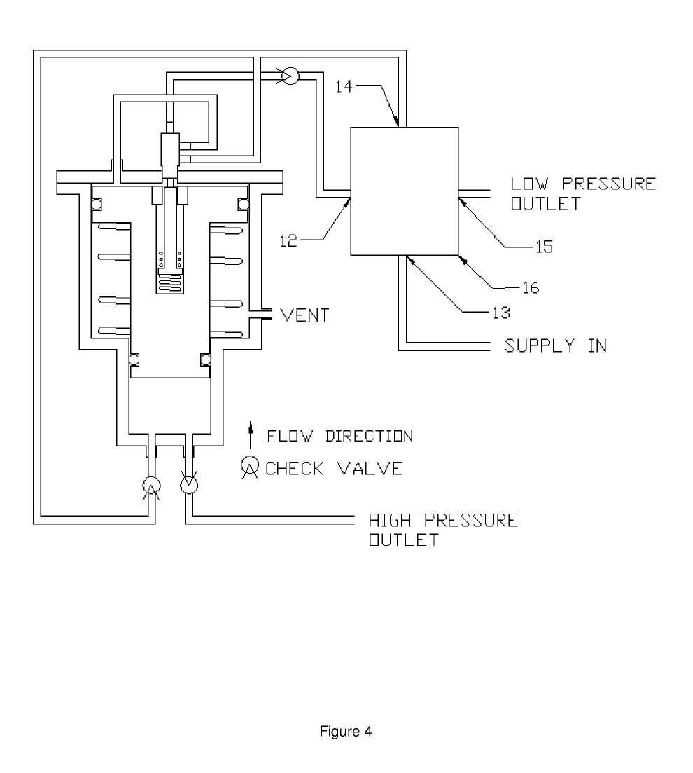

FIG. 4 shows the heat exchanger, item 16, moved to the supply gas inlet to the intensifier circuit. FIG. 4 is a sectional view of the intensifier. Flow of the supply gas is from item 13 to item 14. Flow of the low pressure gas is from item 12 to item 15.

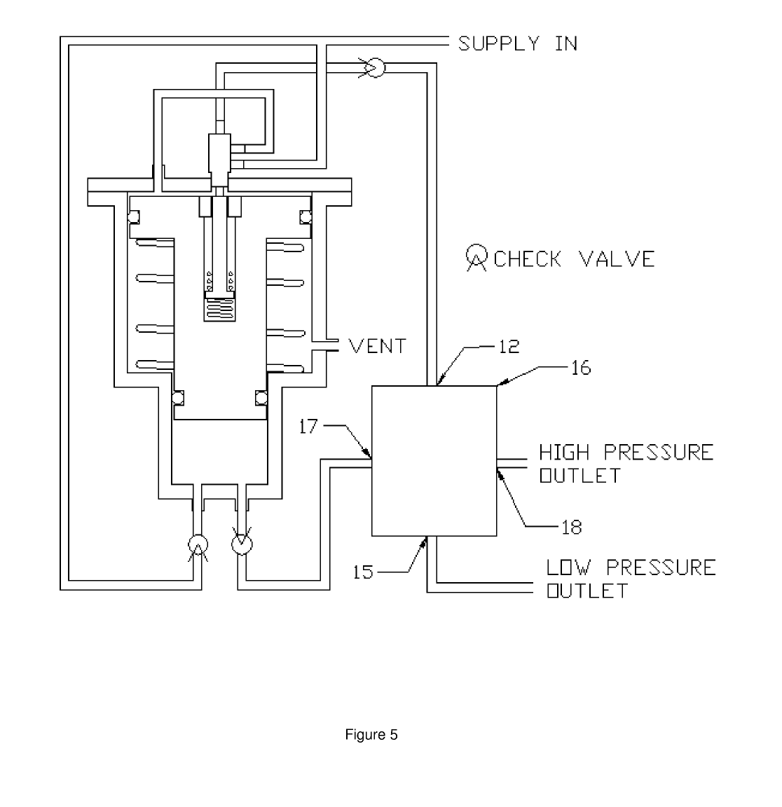

FIG. 5 shows the heat exchanger, item 16, moved to the high pressure gas section of the intensifier circuit. FIG. 5 is a sectional view of the intensifier. Flow of the high pressure gas is from item 17 to item 18. Flow of the low pressure gas is from item 12 to item 15.

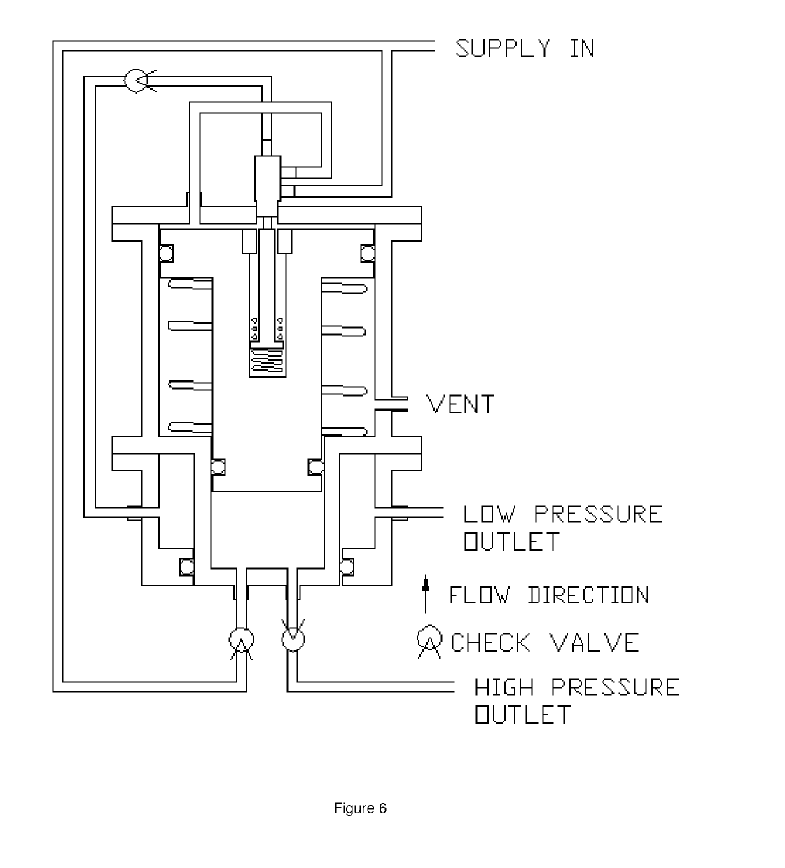

FIG. 6 shows the heat exchanger integrated into the pressure intensifier. FIG. 6 is a sectional view of the intensifier.

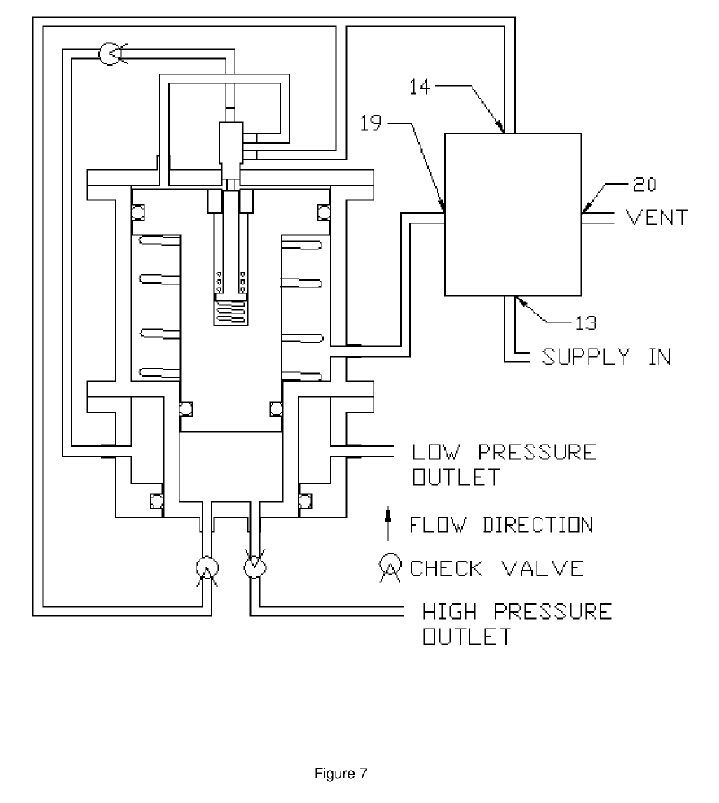

FIG. 7 shows heat exchanger using the center chamber to provide cooling gas flow through the heat exchanger. FIG. 7 is a sectional view of the intensifier. Supply gas flows from item 13 to item 14. Vent gas flows between items 19 and 20.

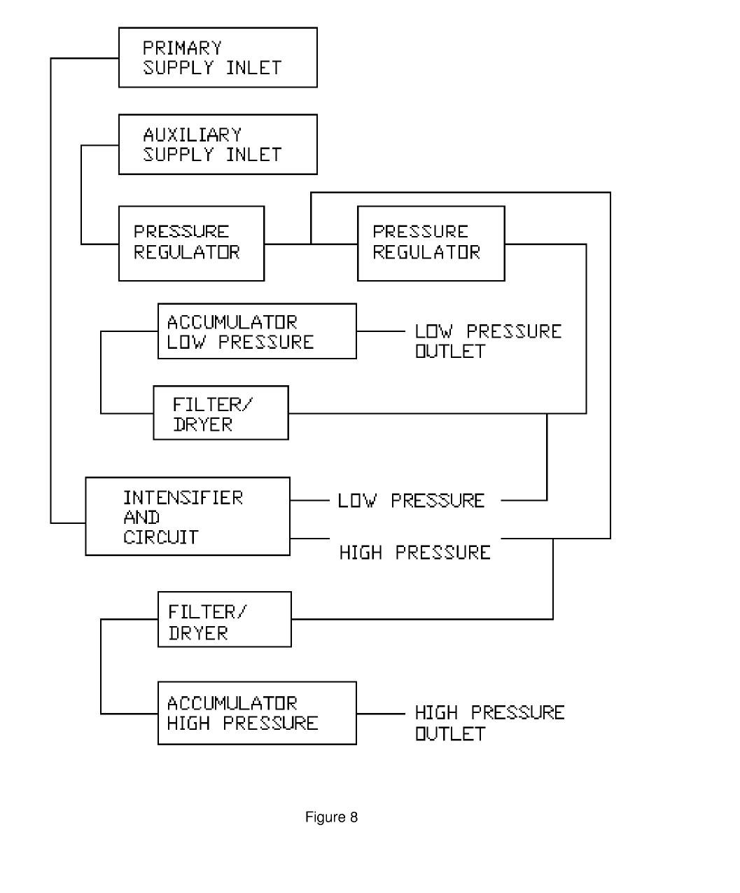

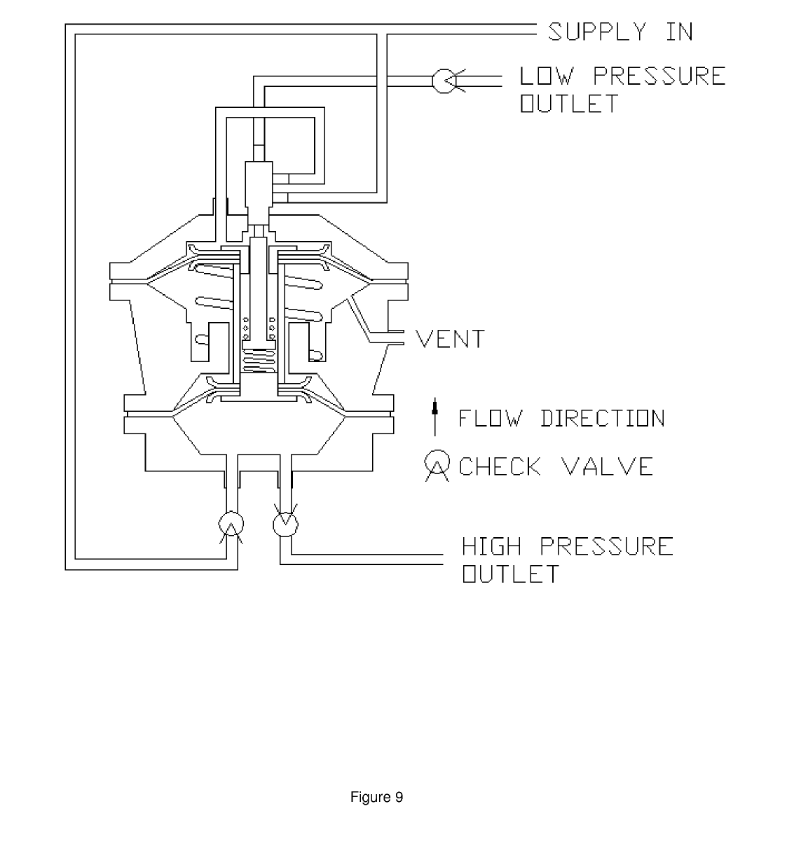

FIG. 8 is a diagram of the components in the system.

FIG. 9 is a diaphragm type pressure intensifier. FIG. 9 is a sectional view of the intensifier. The diaphragms replace the piston assembly of FIG. 1 and FIG. 2.

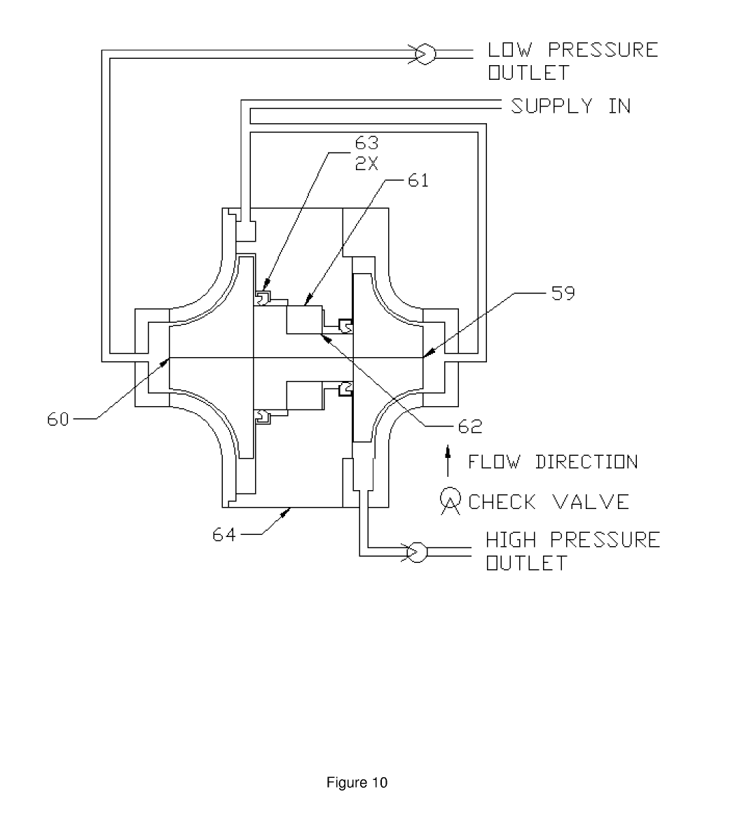

FIG. 10 is a turbine and a compressor pressure intensifier. FIG. 10 is a sectional view of the intensifier. Supply gas is connected to the compressor and turbine inlets. High pressure gas exits from the compressor and low pressure gas exits from the turbine. Item 59 is the compressor. Item 60 is the turbine. Item 61 is the bearing. Item 62 is the shaft. Item 63 is the seal, there are two seals in the assembly. Item 64 is the housing.

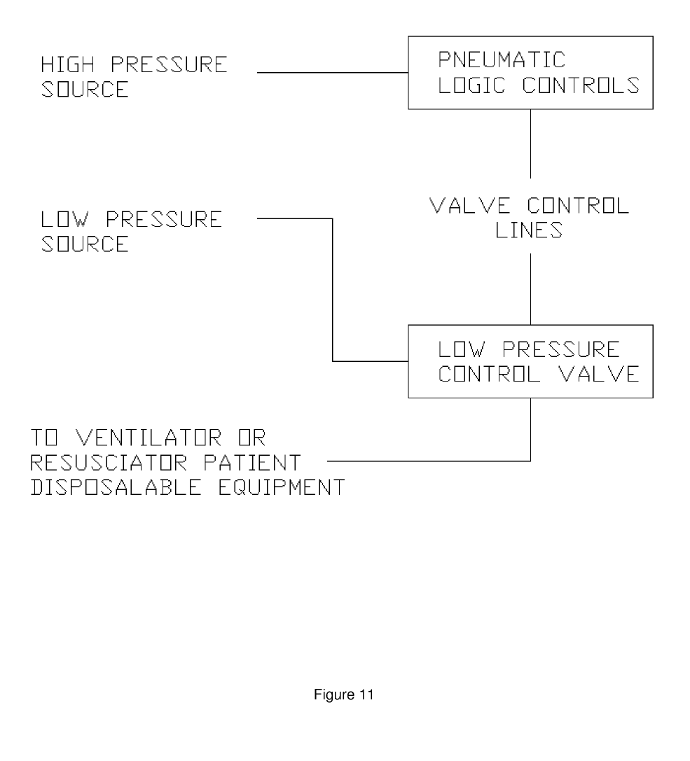

FIG. 11 is a diagram showing the hook up of the lines from the intensifier circuit to the low pressure control valve and the pneumatic logic controls. The outlet flow is to the ventilator or resuscitator disposable equipment hookup.

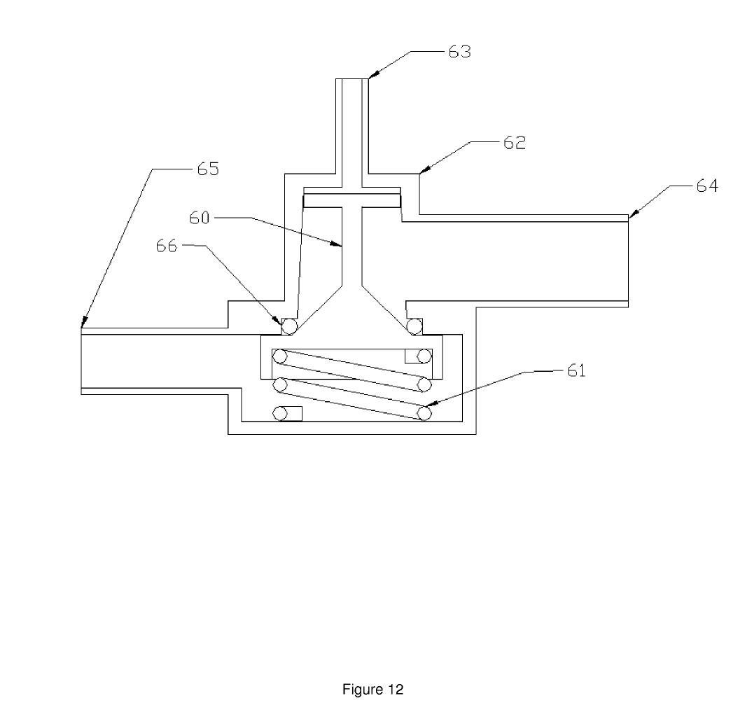

FIG. 12 is a low pressure proportional control valve. FIG. 12 is a sectional view of the control valve. Item 65 is the inlet for the low pressure supply. Item 66 is the seal for the poppet item 60. Item 61 is a spring which forces the poppet into the sealed position shown. Item 62 is the valve housing, the housing is tapered in the area where the upper part of the poppet, item 60, moves. Item 64 is the outlet of the valve. Item 63 is the inlet for logic control signal line. The housing, item 62, and the upper part of the poppet, item 60, form a variable annular orifice. Flow of gas in to item 63 applies force to the poppet, item 60, pushing the poppet down. A force balance will be achieved relative to the flow of gas from the control logic and the position of the poppet which will proportional control the flow of low pressure gas through the lower section of the poppet.

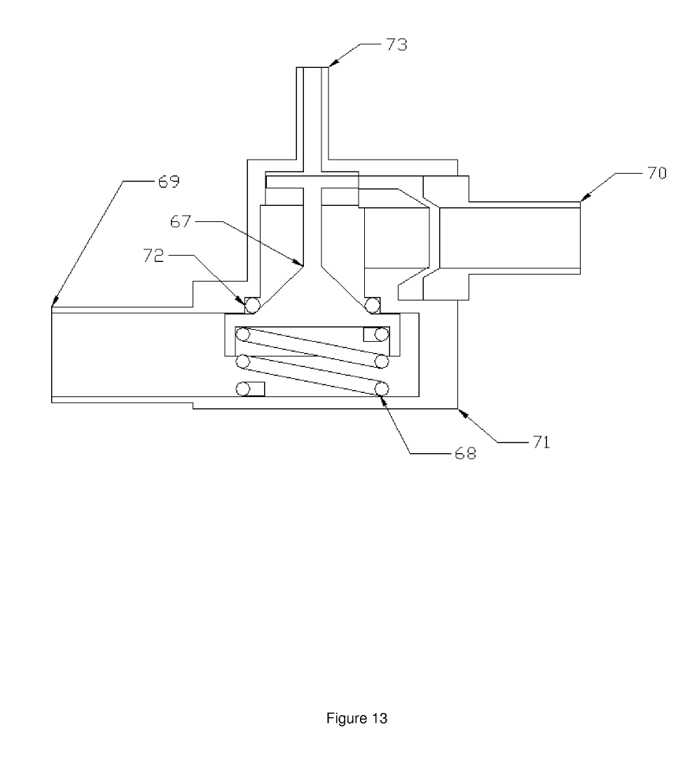

FIG. 13 is a low pressure proportional control valve. FIG. 13 is a sectional view of the control valve. Item 69 is the inlet for the low pressure supply. Item 72 is the seal for the poppet item 67. Item 68 is a spring which forces the poppet into the sealed position shown. Item 71 is the valve housing. Item 70 is the outlet of the valve. Item 73 is the inlet for logic control signal line. The housing, item 71, and the upper part of the poppet, item 60, form a variable orifice. Flow of gas in to item 73 applies force to the poppet, item 67, pushing the poppet down. A force balance will be achieved relative to the flow of gas from the control logic and the position of the poppet which will proportional control the flow of low pressure gas through the lower section of the poppet. A venturi tube is formed by the housing, item 71, and valve outlet, item 70. This provides a method for increasing the flow of gas through the low pressure proportional valve.

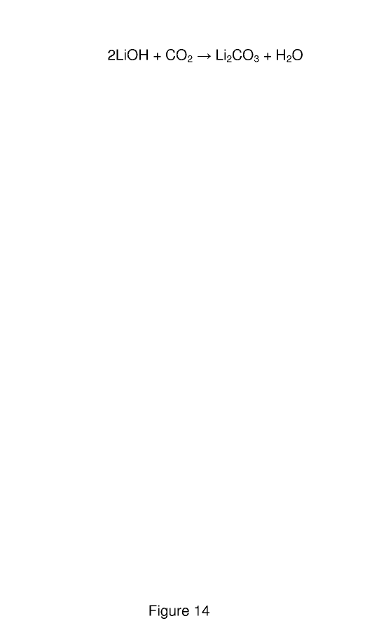

FIG. 14 is the lithium hydroxide and carbon dioxide reaction. Lithium hydroxide is used widely as absorbent of carbon dioxide. A filter bed for use in a chemical oxygen generator can easily be produced by using a water and lithium hydroxide monohydrate wash on the ceramic filter media. Removing the excess solution and drying the product at temperatures exceeding 212 degrees Fahrenheit at which time lithium hydroxide monohydrate will give release the water forming lithium hydroxide.

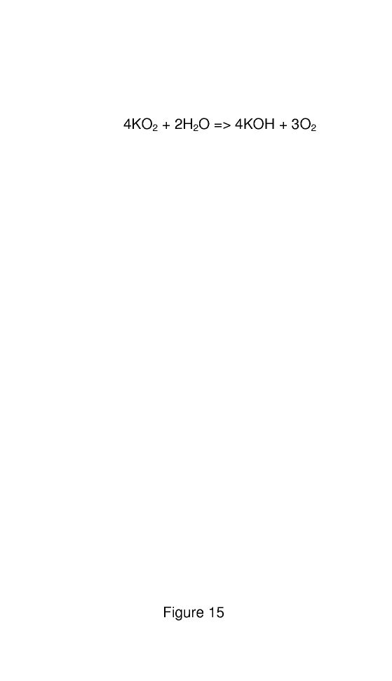

FIG. 15 is the potassium supper oxide and water reaction. Potassium supper oxide is widely used to absorb water and carbon dioxide in a gas stream. Potassium supper oxide readily reacts with water and carbon dioxide and can be purchased reduced to optimum particle size from suppliers

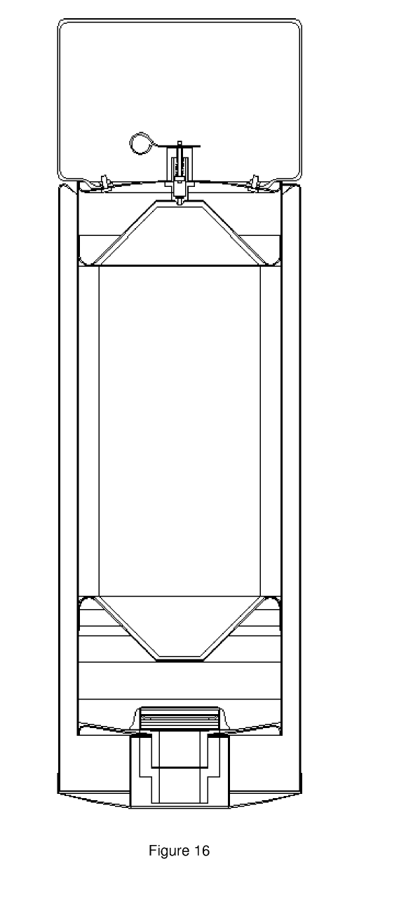

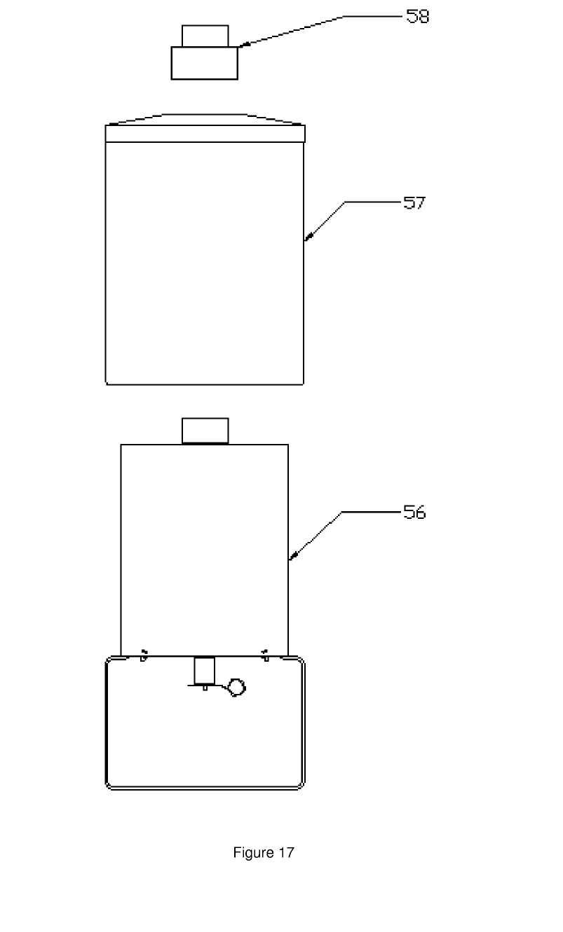

FIG. 16 is an assembled chemical oxygen generator specifically designed for use with a ventilator or resuscitator. FIG. 16 is a sectional view of the chemical oxygen generator. The section is taken along the plane of the handle fasteners and the central axis of the generator tube. FIGS. 17 through 27 use the same sectioning plane.

FIG. 17 shows the assembled chemical oxygen generator cartridge item 56, the assembled heat shield, item 57, and the connector fastener, item 58. Item 57 slides over item 57 and is held in place by item 58.

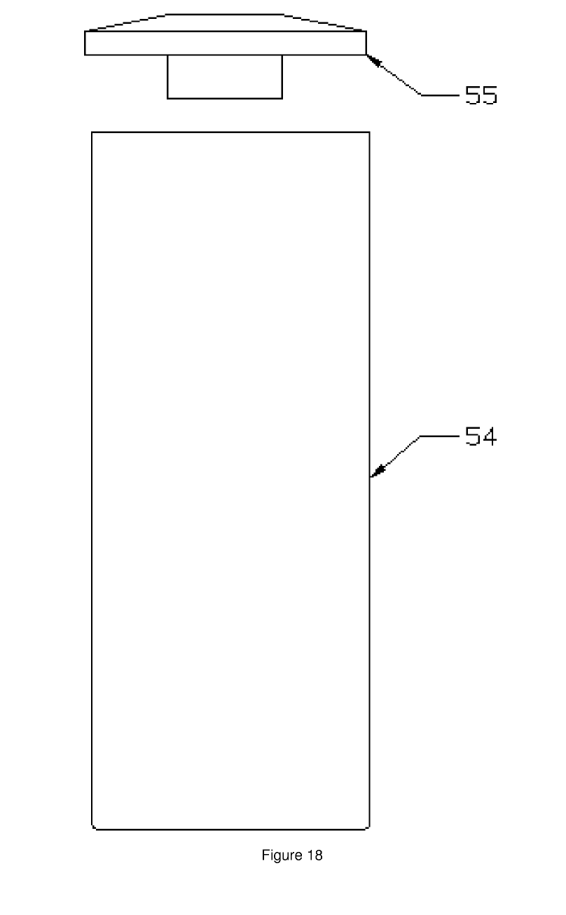

FIG. 18 shows the part that make up the heat shield item 57 of FIG. 17. Item 54 is the barrel of the heat shield and item 55 is the end cap of the heat shield. Item 54 is open at the bottom to allow the generator cartridge, item 56, of FIG. 17 to be place inside the assembled heat shield.

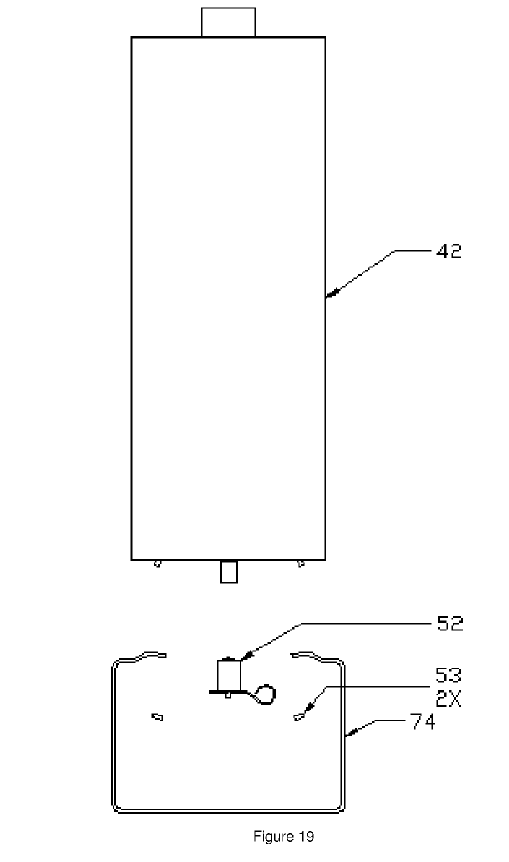

FIG. 19 shows the handle, item 74, the fasteners, items 53, and the initiation mechanism, item 52. The fasteners hold the handle in place.

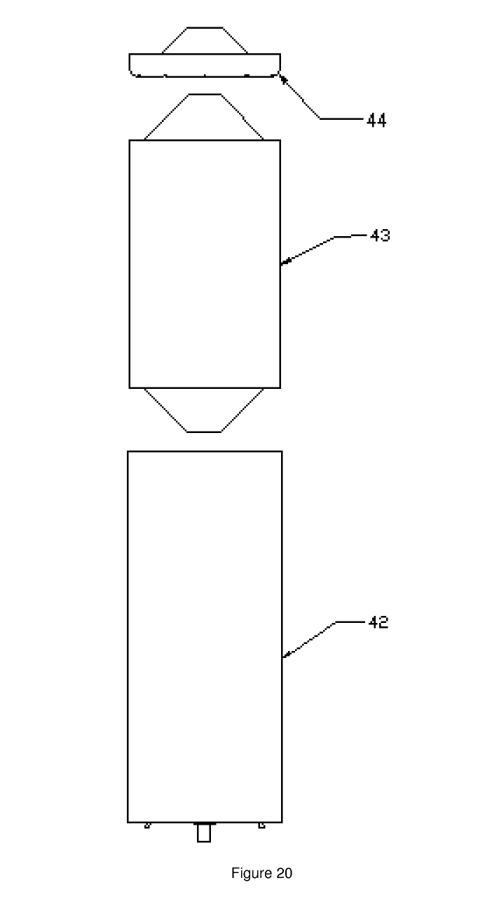

FIG. 20 shows chemical oxygen generator can, item 42, the chemical oxygen generator core with the insulator in place, item 43, and the exit end core locator, item 44. The exit end core locator, item 44, is a press fit into the generator can, item 42, and spot welded into place. The core locator has a ring of holes near the outer edge which allows a gas flow. The core locator has a sharp edge at the largest diameter. The outer surface of the core locator is tapered, one half degree, with the largest diameter at the sharp edge end. The sharp edge of the core locator with hold the chemical oxygen generator core in place due to the shape edge of the core locator cutting into the generator can. The tube end cap and the generator cartridge tube are of similar materials to allow the assembly to be TIG welded together.

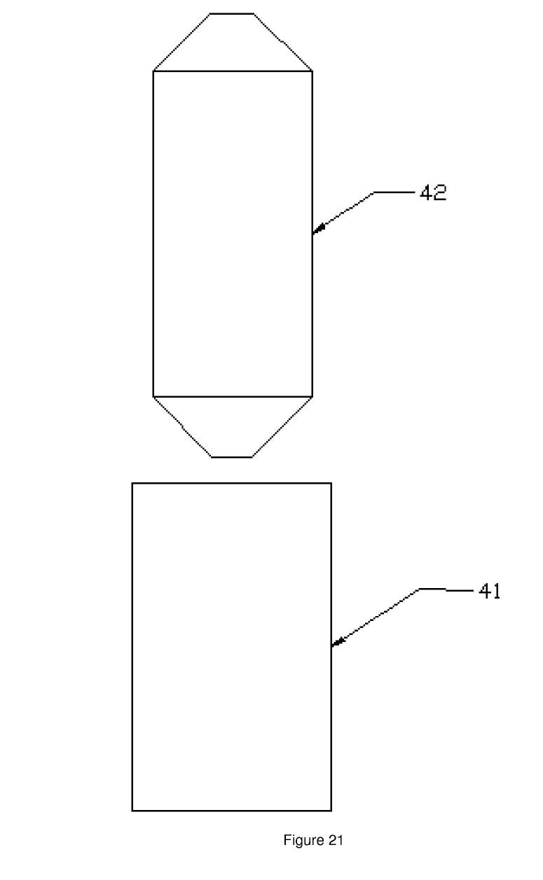

FIG. 21 shows the chemical generator core, item 42, and the insulator, item 41. The core is a press fit into the insulator.

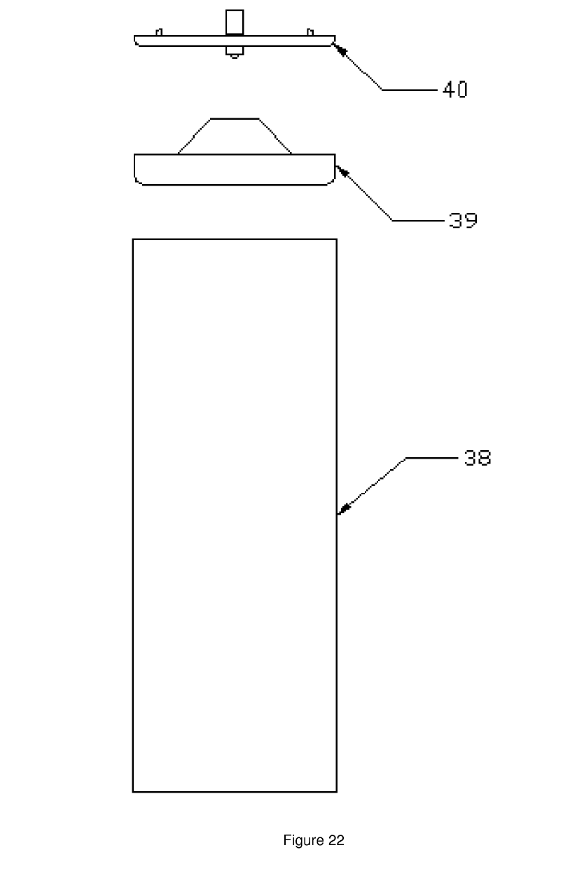

FIG. 22 shows the generator cartridge tube, item 38, the initiator end core locator, item 39, and the initiator end cap assembly, item 40. The core locator is similar to the design of item 44 of FIG. 44, with the exception of only one hole in the center of the locator. Item 40 is a press fit into the generator cartridge tube, item 38, and spot welded into place. The tube end cap and the generator cartridge tube are of similar materials to allow the assembly to be TIG welded together.

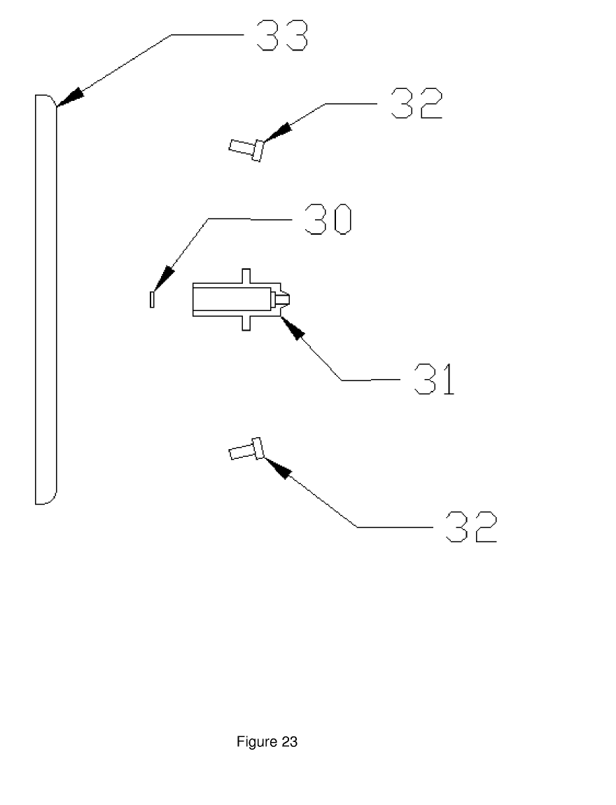

FIG. 23 shows the parts that make up the initiator end cap assembly. Item 33 is the end cap. Item 32 is a fastener used in FIG. 19. Item 31 is the initiator tube. Item 30 is a magnum rifle primer. Item 30 is pressed into item 31 and staked in place with a circular pattern. Item 32 is silver soldered or brazed on the end cap, item 33.

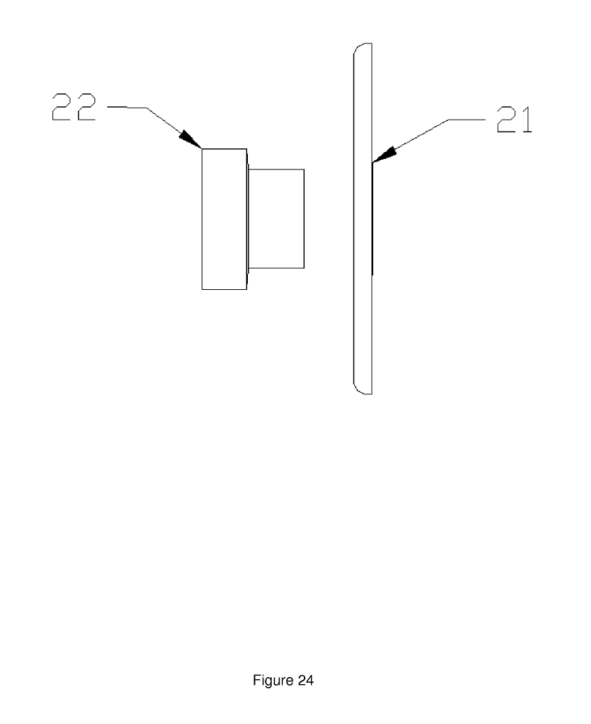

FIG. 24 shows the exit end cap, item 21 and the particle filter holder, item 22. These parts are silver soldered or brazed together.

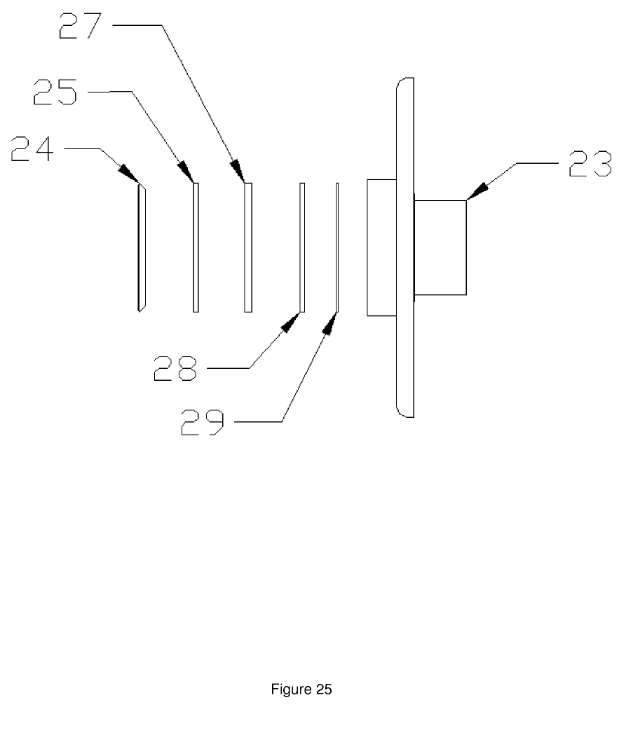

FIG. 25 shows the parts of the exit end cap particle filter assembly and the exit end cap. Item 29 is a rupture disk. Items 25 and 28 are screens used to sandwich the particle filter media, item 27. Item 24 is a ring designed to be a press fit in to the particle filter holder. Item 29 is silver soldered or brazed into the particle filter holder.

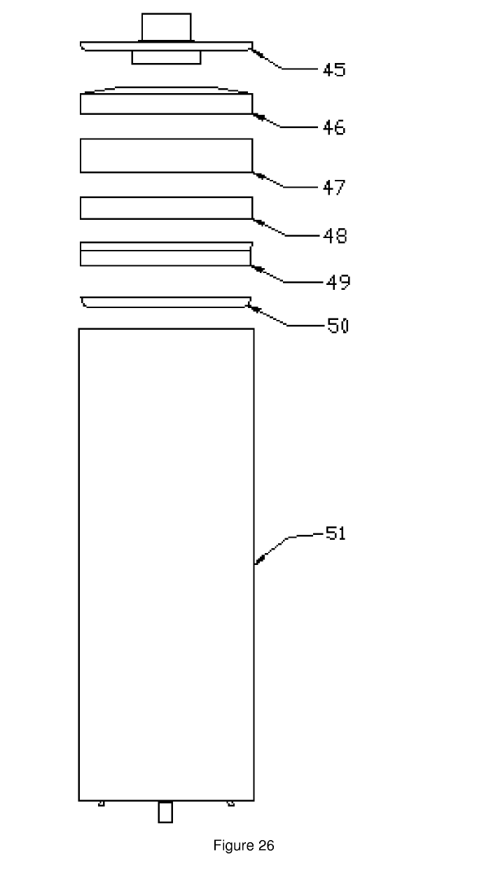

FIG. 26 shows the materials used to assemble the filter of the chemical generator cartridge. Item 50 is a ceramic fiber particle filter ring. Item 49 is a filter bed of hopcalite. Item 48 is a ceramic fiber media which may be impregnated with lithium hydroxide. Item 47 is a filter bed of potassium super oxide. Item 46 is a ceramic fiber pad used to compress the filter chemical filter system. Item 45 is the fully assembled exit end cap of FIG. 25. Item 51 is the cartridge tube sub assembly of FIG. 20.

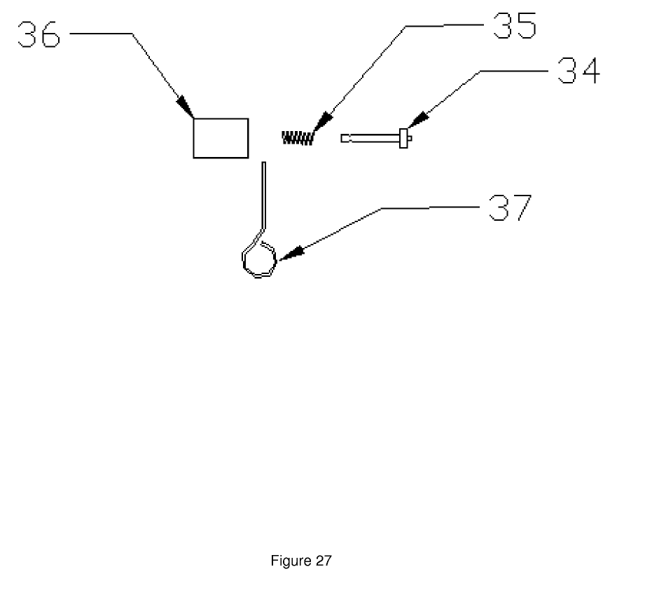

FIG. 27 shows the parts of the initiation mechanism. Item 37 is the release pin. Item 36 is the initiator cap. Item 35 is the initiator spring. Item 36 is the initiator pin. Item 35 and 34 are placed in item 36 and the spring, item 35, is compressed allowing item 37 to be place in a hole through item 34.

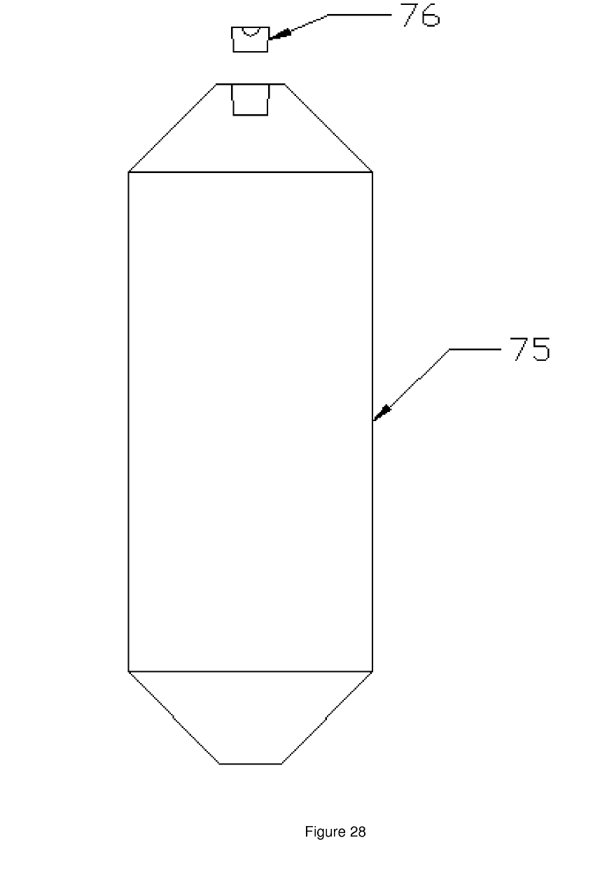

FIG. 28 shows the initiator pellet, item 76 and the main body of the chlorate candle, item 75. The cavity of the main body, item 76, has taper of 1.5 degrees. The initiator pellet, item 76, has a taper of 1.5 degrees. The diameters of the tapers are such that the pellet when pressed into the main body of the chlorate candle the pellet will be recessed in candle.

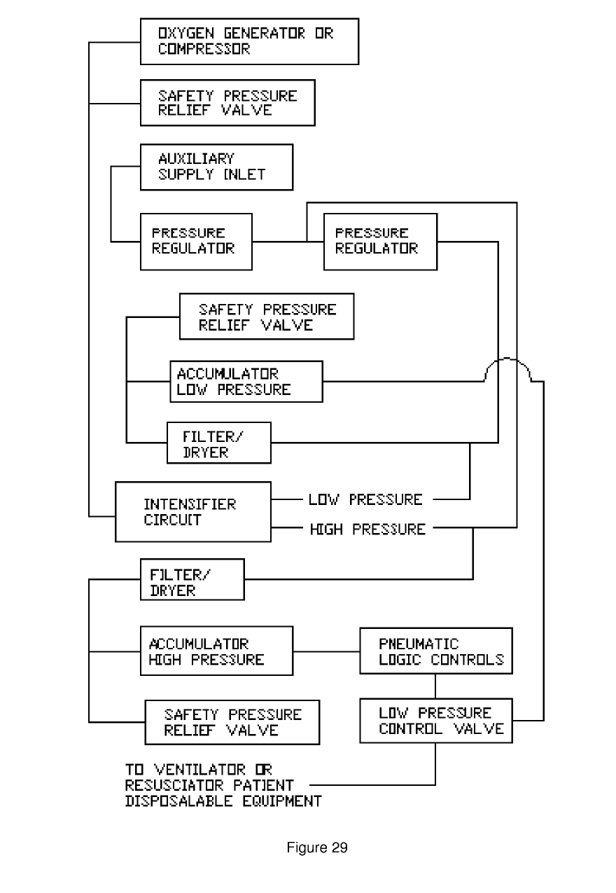

FIG. 29 is a diagram of the invention showing the information of FIG. 8, FIG. 11, and the intensifier circuit; with three safety pressure relief valves added.

### SEQUENCE LISTING

Not Applicable

1. The intensifier operates by mechanical means, using only the energy supplied by the gas source.

2. A chemical gas generator or compressor provides the primary gas source to operate a gas pressure intensifier of claim 1.

3. Using a gas pressure intensifier, of claim 1, with the gas generator, of claim 2, reduces the weight of the generator pressure vessel.

4. A two path heat exchanger and the intensifier, of claim 1, can be integrated in to one assembly.

5. The gas pressure intensifier, of claim 1, increase the amount of usable gas delivered to a ventilators or resuscitators over a high pressure gas generator due to the amount of gas left in the high pressure container when minimum operating pressure is reached in the high pressure gas generator.

6. The design of the intensifier assembly of claim 1 allows the immediate charging of the high pressure accumulator to the pressure of the intermediate pressure source.

7. A variable annular orifice is created by placing a disk, or other shape, in a cone shaped tube. A spring holds the disk in the small end of the cone shaped tube. Gas flows from the small diameter end to the large diameter in. The flow of gas will push the disk towards the large end of the tube until a force balance is achieved. The greater the flow the farther the disk will be from the small end of the cone shaped tube.

8. A variable orifice is created by placing a disk in a straight tube with a hole in the side of the tube. A stop and a spring are used to hold the disk in a position covering the hole in the side of the tube. When pressure is applied to the entrance end of the tube the pressure will push the disk thereby opening a passage for gas flow. Increasing the gas pressure will increase the area of the hole that is exposed.

9. Variable gas flow from a pneumatic logic controller can be used to proportionally control the flow of a gas stream by means of a variable orifice, of claim 7 or 8, mechanically connected to a proportional valve.

10. The exhaust from the orifice of claims 7 and 8 can be used to power a venturi tube.

11. The venturi tube of claim 10 can be designed in the proportional control valve of claim 9.

12. The design of the proportional valve, of claim 9, adds the flow from pneumatic control logic to the flow from the low pressure valve within the proportional valve assembly.

13. Using a like taper to form the cavity in the main body of the chlorate candle and a matching tapered mold to form the pellet allows the pellet to be pressed into the main body of the chlorate candle.

14. Manufacturing the chlorate candle and the initiator pellet, of claim 13, of the chlorate chemical oxygen generator separately allows each component to be tested separately for performance.

15. A filter bed of a supper oxide removes water from the gas stream of a chlorate candle chemical oxygen generator of claim 13.

16. Reducing the temperature of the high pressure gas stream to less than 200 degrees Fahrenheit allows the use of desiccants such as lithium hydroxide or a molecular sieve to remove water from the gas streams.
